(19) United States
(12) Patent Application Publication
(10) Pub. No.: US 2026/0096895 A1
(43) Pub. Date: Apr. 9, 2026

(54) Tibial Insert

(71) Applicant: Howmedica Osteonics Corp., Mahwah, NJ (US)

(72) Inventors: Damon J. Servidio, Towaco, NJ (US); Chad Bean, Norton, MA (US)

(21) Appl. No.: 18/909,071

(22) Filed: Oct. 8, 2024

(51) Int. Cl. A61F 2/38; A61F 2/30

(52) U.S. Cl. CPC A61F 2/389; A61F 2/3859; A61F 2002/30604

(57) ABSTRACT

Disclosed herein are joint implants with sensors. A tibial implant according to the present disclosure can a tibial baseplate configured to contact a tibia, and a tibial insert configured to contact a femoral implant. The tibial insert can include an articular surface configured to contact a corresponding femoral articular surface of the femoral implant, at least one sensor and a battery disposed within a void of the tibial insert, and a detachable case configured to seal an opening of the void. A thickness of the tibial insert between the detachable case and the articular surface of the tibial insert may be 2 mm or more.

### FIELD OF INVENTION

The present disclosure relates to knee implants, and particularly to modular knee implants with sensors.

### BACKGROUND OF THE INVENTION

Total knee arthroplasty (TKA) typically involves the implantation of a knee joint prosthesis that includes a femoral component and a tibial component. The tibial component generally consists of a tibial insert, a tibial baseplate, and a tibial stem. Recent innovations in TKA have introduced the integration of sensors into these components, particularly within the tibial insert, baseplate, or stem, to monitor joint performance and provide valuable data for patient care.

However, integrating sensors into the tibial insert poses significant challenges. One of the primary issues is the limited space available within the tibial insert to house sensors and electronic components. These components must be strategically placed to ensure they are protected from the high loads exerted by the femoral implant during knee movement, especially in areas subjected to repetitive stress. Inadequate placement or design could lead to component failure or premature wear of the implant. In some cases, inadequate space within the insert limits sensor placement options, leading to compromised sensor functionality.

Therefore, there exists a need for improved knee implants with sensors.

### BRIEF SUMMARY OF THE INVENTION

Disclosed herein are modular joint implants with sensors, and more specifically a modular tibial implant that optimizes sensor placement while maintaining the necessary strength and durability to withstand the loads imposed during knee articulation.

In accordance with an aspect of the present disclosure, a knee implant is provided. A knee implant according to this aspect, may include a femoral implant configured to be coupled to a femur, and a tibial implant configured to be coupled to a tibia. The tibial implant may include a tibial insert disposed between the femoral implant and a tibial baseplate. The tibial insert may include an articular surface configured to contact a corresponding femoral articular surface, at least one sensor and a battery disposed within a void of the tibial insert, and a detachable case configured to seal an opening of the void. A thickness of the tibial insert between the detachable case and the articular surface of the tibial insert may be 2 mm or more.

Continuing in accordance with this aspect, a thickness of the tibial insert between the void and the articular surface of the tibial insert may be 2 mm or more.

Continuing in accordance with this aspect, a proximal portion of the case may define a first radius and the articular surface of the tibial insert may define a second radius. The first radius may be proportional to the second radius.

Continuing in accordance with this aspect, the at least one sensor and the battery may be disposed within the void in a central region of the tibial insert between a medial central region and a lateral central region.

Continuing in accordance with this aspect, the at least one sensor may include a Hall sensor and the femoral implant may include a magnet. The Hall sensor may be configured to track a location of the magnet. The at least one sensor may include a plurality of sensors. The plurality of sensors may include at least one load sensor. The plurality of sensors may include a temperature sensor, a pressure sensor, and a pH sensor. The at least one battery may include a plurality of batteries. The tibial insert may include a printed circuit board assembly, a processor, a charging coil, and an antenna located away from a medial central region and a lateral central region.

Continuing in accordance with this aspect, the detachable case may be configured to hermetically seal the opening.

In accordance with another aspect of the present disclosure, a tibial implant is provided. A tibial implant according to this aspect may include a tibial baseplate configured to contact a tibia, and a tibial insert configured to contact a femoral implant. The tibial insert may include an articular surface configured to contact a corresponding femoral articular surface of the femoral implant, at least one sensor and a battery disposed within a void of the tibial insert, and a detachable case configured to seal an opening of the void. A thickness of the tibial insert between the detachable case and the articular surface of the tibial insert may be 2 mm or more.

Continuing in accordance with this aspect, a thickness of the tibial insert between the void and the articular surface of the tibial insert may be 2 mm or more.

Continuing in accordance with this aspect, a proximal portion of the case may define a first radius and the articular surface of the tibial insert may define a second radius. The first radius may be proportional to the second radius.

Continuing in accordance with this aspect, the at least one sensor and the battery may be disposed with the void in a central region of the tibial insert between a medial central region and a lateral central region.

Continuing in accordance with this aspect, the at least one sensor may include a Hall sensor configured to track a location of a magnet of the femoral implant. The at least one sensor may include a plurality of sensors, the plurality of sensors including at least one load sensor. The plurality of sensors may include a temperature sensor, a pressure sensor, and a pH sensor. The at least one battery may include a plurality of batteries. The tibial insert may include a printed circuit board assembly, a processor, a charging coil, and an antenna located away from a medial central region and a lateral central region.

Continuing in accordance with this aspect, the detachable case may be configured to hermetically seal the opening.

### BRIEF DESCRIPTION OF THE DRAWINGS

A more complete appreciation of the subject matter of the present disclosure and the various advantages thereof can be realized by reference to the following detailed description, in which reference is made to the following accompanying drawings:

FIG. 1 is a back perspective view of a knee joint implant according to an embodiment of the present disclosure;

FIG. 2 is front perspective view of the knee joint implant of FIG. 1;

FIG. 3 is a top view of the of a tibial insert of the knee joint implant of FIG. 1;

FIG. 4 is cross-sectional view of the knee joint implant of FIG. 1;

FIG. 5 is a cross-sectional view of the tibial insert of FIG. 3;

FIG. 6 is a side cross-sectional view of the tibial insert of FIG. 3, and

FIG. 7 is a cross-sectional view of a tibial insert according to another embodiment of the present disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “load” and “force” will be used interchangeably and as such, unless otherwise stated, the explicit use of either term is inclusive of the other term. Similarly, the terms “magnetic markers” and “markers” will be used interchangeably and as such, unless otherwise stated, the explicit use of either term is inclusive of the other term.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior” means closer to the head, and the term “inferior”means more distant from the head.

The present disclosure provides a modular tibial implant for use in TKA according to an embodiment. The tibial implant is designed to address the challenges associated with integrating sensors and electronic components within the implant while maintaining structural integrity. The tibial implant generally includes a tibial insert, a tibial baseplate, and a tibial stem, with the option to incorporate a sensor module into any of these components. An aspect of this embodiment is the modular design of the tibial insert, which includes a dedicated slot and opening for securely housing sensors, batteries, and other electronic components. These components can be arranged within hollow volumes of the tibial insert, strategically located to avoid high-load areas while maximizing the strength and wear resistance of the implant. A modular case is designed to fit into the tibial insert via a posterior assembly that ensures a soft tissue-friendly fit and reduces pressure on surrounding tissues. This design enhances the performance of the posterior cruciate ligament (PCL) by allowing it to move freely without obstruction during knee articulation.

The tibial baseplate is designed to accommodate the tibial insert with secure locking mechanisms that prevent micromotion and ensure a stable connection under normal knee loading conditions. The baseplate can include features such as an anterior locking wire and a center island for secure attachment to the tibial insert. The tibial stem provides further stability by anchoring the implant to the tibia, allowing for efficient load transfer, and minimizing the risk of implant loosening.

Disclosed herein are various embodiments of the sensor module that can be customized to meet patient-specific needs. The sensor module may include a range of sensors such as load sensors, Hall effect sensors, inertial measurement units (IMUs), pH sensors, and temperature sensors. These sensors are capable of monitoring joint performance in real-time, providing valuable data on loading conditions, ligament tension, and implant stability. The modular nature of the implant allows for convenient assembly and customization during surgery. A surgeon can select the appropriate tibial insert size and sensor module configuration based on the patient's anatomy and clinical needs. Once the components are assembled, the tibial insert is hermetically sealed to protect the electronic components from external contaminants and to ensure long-term durability.

By addressing the spatial and structural challenges of sensor integration, the present disclosure provides a tibial implant that enhances knee function, reduces the risk of complications, and improves patient outcomes. The modular design simplifies the manufacturing process, improves sterilization efficiency, and offers enhanced flexibility in clinical use.

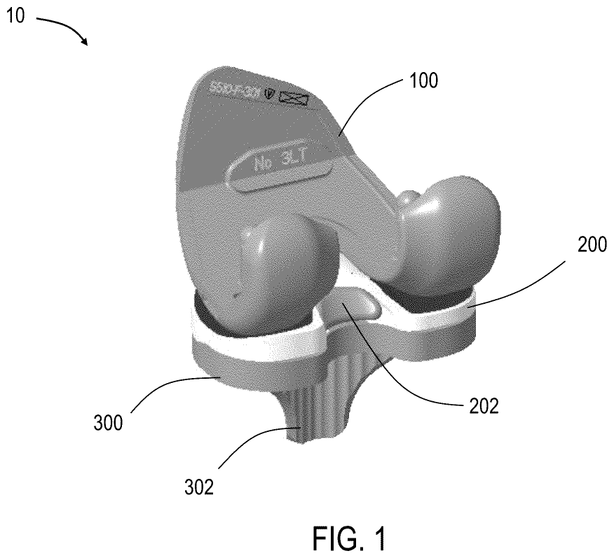

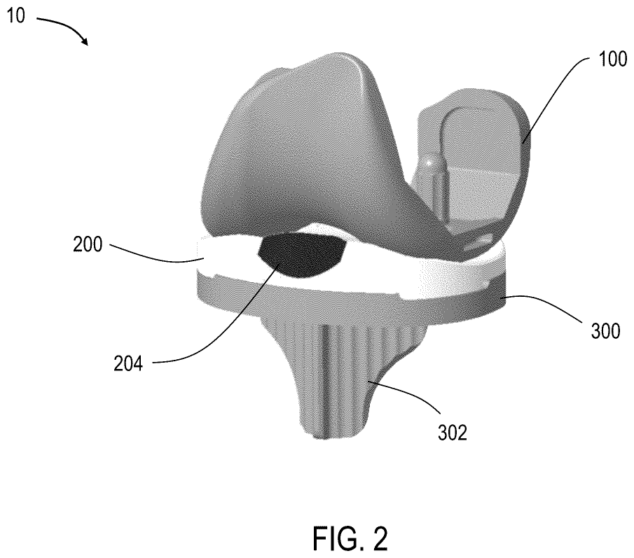

FIGS. 1 and 2 illustrate perspective views of a knee joint implant 10 in accordance with an embodiment of the present disclosure. The knee joint implant includes a femoral implant 100 and a tibial implant. The tibial implant consists of a tibial insert 200, designed to articulate with the femoral implant 100, a tibial baseplate 300, and a tibial stem 302 extending distally into the tibia. Various sensors and electronic components can be integrated into knee joint implant 10 to monitor performance.

In one embodiment, femoral implant 100 includes a medial encoder track positioned on the medial side and a lateral encoder track located on the lateral side. These tracks can be fully or partially embedded within the femoral implant on the respective sides. The encoder tracks can comprise different structures, such as magnetic tape of varying lengths or discrete magnetic markers. The resolution of the encoder tracks can be customized to suit the precision required for measuring parameters like joint displacement, rotation, and slip.

Tibial insert 200 can include a medial read head and a lateral read head, which are configured to detect and measure magnetic flux density from the medial and lateral encoder tracks. These read heads can utilize suitable magnetometers, such as Hall effect sensors, to capture the magnetic flux density. Additionally, tibial insert 200 can be equipped with a data transmitter, such as an antenna, to wirelessly transmit the measured knee joint parameters via Bluetooth or similar wireless protocols to an external device, such as a smartphone, tablet, monitor, or network, enabling real-time performance monitoring.

A battery can be housed within the tibial insert 200 to power the various sensors and electronic components within knee joint implant. Other sensors, including temperature sensors, pressure sensors, accelerometers, gyroscopes, magnetometers, pH sensors, and others, can be incorporated into the knee joint implant 10.

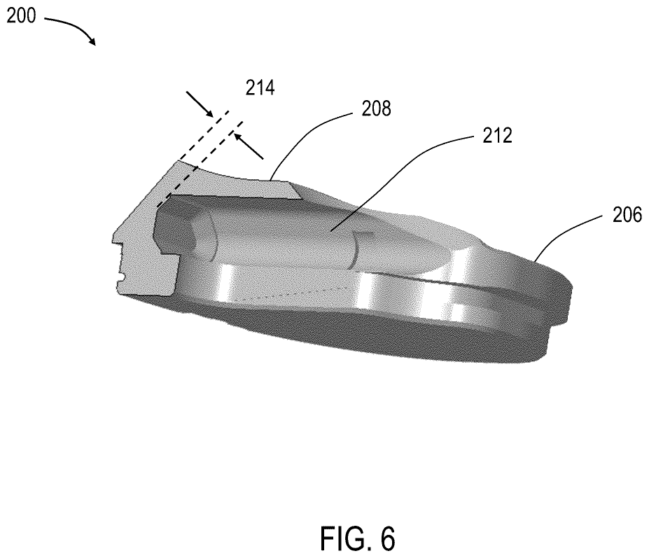

Tibial insert 200 is provided with a modular case 202, specifically configured to securely fit into the tibial insert via a slot 212 with an opening, as depicted in FIG. 6. Modular case 202 is configured for posterior insertion into the opening of the tibial insert 200. This posterior placement is necessary for maintaining the structural integrity of the insert while allowing for easy assembly and alignment of sensor components within the tibial insert. The biomechanics of the knee joint require careful consideration of the ligaments during various degrees of flexion and extension. Specifically, the anterior cruciate ligament (ACL) is taut from full extension to approximately 45 degrees of flexion, exerting an anterior force on the femur. Conversely, the posterior cruciate ligament (PCL) becomes taut beyond 45 degrees of flexion, pulling the femur posteriorly as flexion progresses. Most knee prostheses are designed to accommodate clearance for the PCL by simulating the natural PCL notch in the tibia, particularly at the 45-degree point when the PCL becomes taut. Additionally, the PCL is relaxed from 45 degrees to full extension, which provides opportunities for noncontinuous surface designs in this area of the tibial insert.

In particular, the PCL notch of the tibial insert offers an ideal location for assembling the sensor case 202. This area, located posteriorly, avoids interference with high-load surfaces that bear the forces exerted by the femoral implant during knee articulation. Since the PCL becomes taut during deeper flexion, this posterior location provides a secure and protected space for the sensor case without compromising the structural integrity of the tibial insert. The femoral implant articulates with the tibial insert during flexion, and the load-bearing surface follows a well-defined path. The area beneath this load path must adhere to FDA guidance, requiring sufficient thickness to support the forces generated during flexion and extension. Consequently, material cannot be removed from this critical load-bearing area to accommodate sensor components, making the posterior region an optimal placement for the modular case.

On the other hand, the anterior tibial insert, specifically the patellar relief feature, is unsuitable for sensor assembly. During flexion, the patella tilts and shifts in the sagittal plane at an angle of approximately 35 degrees. The loading of the patellar tendon increases significantly during deeper flexion, making this area sensitive to any interruptions in surface continuity. As a result, the anterior tibial insert surface must remain intact to prevent disruptions in patellar movement and avoid excessive wear or damage.

The posterior assembly of the tibial insert not only ensures a soft tissue-friendly design but also provides a secure fit that minimizes pressure on the surrounding soft tissues. This configuration improves the performance of the PCL by allowing the tibial insert and modular case to be securely positioned where the PCL articulates, without obstructing its natural movement. By maintaining the free movement of the PCL, the posterior assembly reduces the risk of post-operative complications, such as fretting wear, which can arise from metal-to-metal contact between the tibial baseplate and the sealed modular case. Both the baseplate and the case can be manufactured from materials such as titanium or cobalt-chromium to enhance durability and biocompatibility, further ensuring optimal post-operative results.

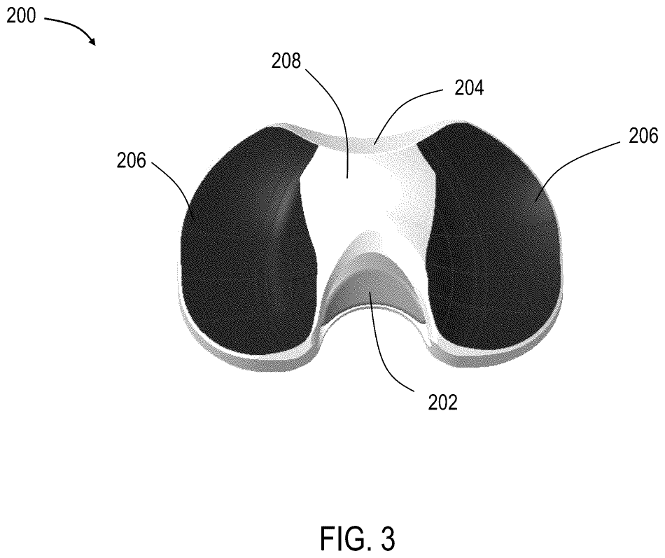

Once modular case 202 is securely attached to the tibial insert 200, as illustrated in FIG. 3, the tibial insert can be affixed to the tibial baseplate 300. FIG. 3 provides a top view of the tibial insert 200, which features an anterior relief 204 and a central ridge 208 that separates the medial and lateral articular surfaces 206. The opening of the slot 212 at the posterior end of the central ridge 208 allows for the insertion of sensors, batteries, and other electronic components into the tibial insert 200. These components are arranged in a manner that avoids high-load areas, ensuring that the structural integrity of the tibial insert remains intact. Once the electronic components are in place, the modular case 202 is inserted and secured, sealing, and locking the components within the tibial insert for long-term protection and functionality.

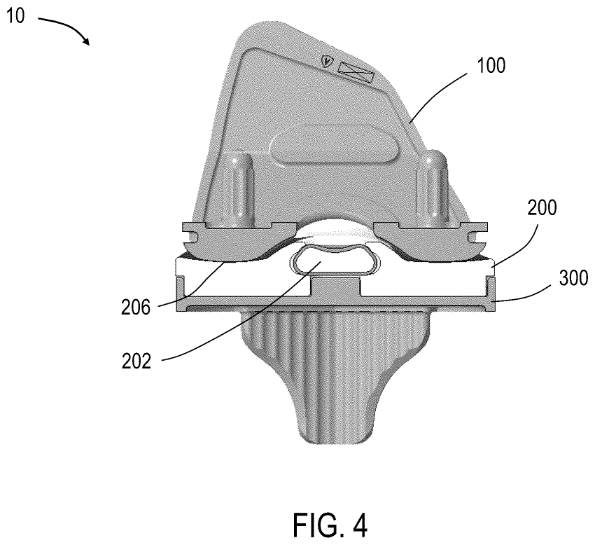

FIG. 4 provides a cross-sectional view of the knee joint implant 10, illustrating the interaction between the femoral implant 100 and the tibial insert 200. As shown in this view, the lateral and medial articular surfaces of the femoral implant 100 are designed to articulate with the corresponding lateral and medial articular surfaces 206 of tibial insert 200. These contact points and surfaces are necessary for ensuring smooth movement and proper load distribution during knee flexion and extension.

In particular, the lateral and medial articular surfaces 206 of the tibial insert 200 are shaped to closely match the geometry of the femoral implant 100. This alignment allows for effective load transfer between the femoral and tibial components, minimizing wear and maintaining the structural integrity of the implant over time. The interaction between these surfaces also ensures that the knee joint operates with a natural range of motion, closely mimicking the biomechanics of a healthy knee. The materials used for the tibial insert's lateral and medial surfaces are selected for their durability, wear resistance, and biocompatibility, ensuring optimal performance under the high loads exerted by the femoral component during normal activities such as walking, standing, and climbing stairs.

As further highlighted in FIG. 3, the areas of contact between the femoral implant 100 and the tibial insert 200 are specifically designed to reduce friction and wear. The medial and lateral surfaces of the tibial insert 200 are located in high-load regions, and as such, are reinforced to maintain their integrity over extended periods of use. The thickness of these areas is governed by FDA guidelines to ensure that the implant can withstand the significant forces generated by the femoral component during knee articulation.

Case 202 is shown sealing the slot 212 within the tibial insert 200 in FIG. 4. The case is securely fitted into the posterior opening of the tibial insert, as described earlier, ensuring that the internal components are hermetically sealed. This sealing mechanism protects the sensitive electronic components from exposure to bodily fluids, wear debris, and external contaminants, which could otherwise compromise their functionality.

Case 202 can be constructed from biocompatible materials that are selected for their ability to resist corrosion, wear, and degradation in the body's environment. The sealing design also ensures that the tibial insert maintains its overall mechanical strength, even with the inclusion of the internal sensor components. The case's positioning at the posterior end of the tibial insert ensures that it remains outside of the primary load-bearing areas, while still providing easy access for assembly and servicing, if necessary.

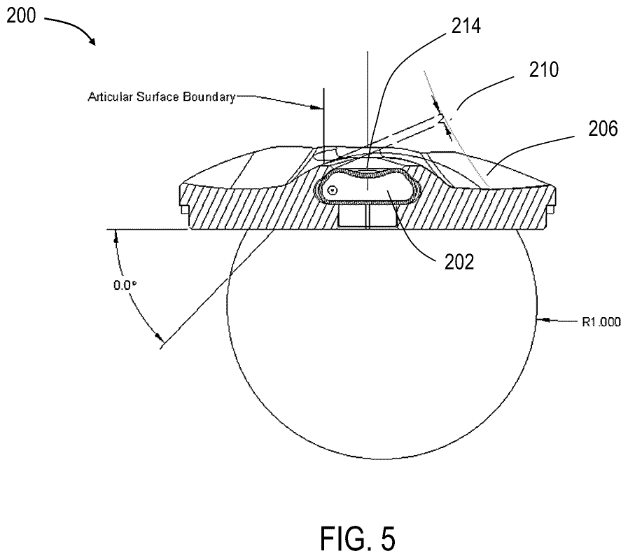

FIG. 5 illustrates a cross-sectional view of tibial insert 200 with case 202, highlighting the key structural features of the case in relation to the tibial insert. In this configuration, the modular case 202 is securely seated within the tibial insert, and the design ensures that a minimum distance of 2 mm is maintained between the case and the articular surface of the tibial insert. This 2 mm clearance is necessary to preserving the structural integrity of the tibial implant, particularly in the high-load areas that are subject to significant forces during knee articulation. By maintaining this 2 mm minimum distance, the design ensures that the load distribution across the tibial insert, especially in the critical medial and lateral articular surfaces, remains consistent with the natural biomechanics of the knee. This clearance prevents any risk of structural compromise, ensuring that the tibial implant can effectively bear the forces exerted by the femoral component during activities such as walking, bending, and standing. The thickness of this area meets FDA guidelines for load-bearing medical implants, further ensuring patient safety and long-term durability.

As illustrated in FIG. 6, slot 212 within the tibial insert is designed to accommodate a modular sensor array which can include a variety of sensor modules and associated electronic components. These components may include various types of sensors, such as Hall effect sensors, load sensors, pH sensors, and temperature sensors, as well as other electronic elements like a battery and an antenna for wireless communication. Slot 212 provides sufficient volume for these components without compromising the overall strength and stability of the tibial insert. A minimum thickness of 2 mm is maintained from a patella relief angle to slot 212 as denoted by a distance 214. In one embodiment, the surface of the tibial insert is concave while the adjacent surface of the modular sensor array is convex which supports maintaining the 2 mm spacing.

Case 202 is configured to integrate seamlessly within the tibial insert while ensuring that sufficient material is maintained in the key load-bearing areas beneath the femoral component's articulation surfaces. The 2 mm minimum thickness at point 210, which corresponds to the boundary between the articular surface and the top corner of the case, is an important design feature. A minimum thickness of 2 mm is maintained uniformly throughout the tibial insert, ensuring that the implant can withstand the repeated loading and stress associated with normal knee movement. The design of the tibial insert, combined with the protective casing for the sensors, ensures that the electronic components remain secure and functional even under high mechanical loads.

Furthermore, the shape of case 202 is optimized for both performance and ease of assembly. The angles of the case increase as the case widens from the proximal to the distal end, as shown in FIG. 6. This widening allows for a better fit within the tibial insert, providing greater stability and ensuring that the case remains securely in place throughout the patient's range of motion. This tapered design also aids in the assembly process, allowing the surgeon to easily insert and position the case without the need for excessive force or complex tools. Additionally, the shape of the case helps to evenly distribute the forces exerted on the tibial insert, minimizing wear, and extending the life of the implant.

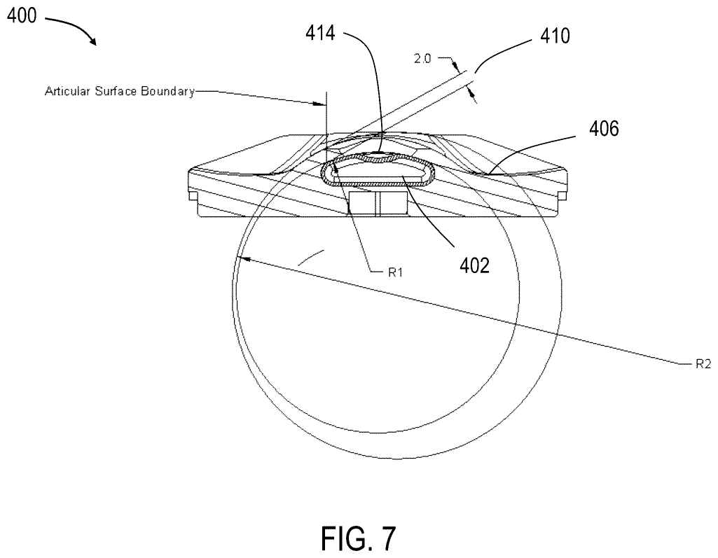

FIG. 7 shows a cross-sectional view of a tibial insert 400 according to another embodiment of the present disclosure. Tibial insert 400 shares several structural similarities with the tibial insert 200, and as such, similar elements are referenced using corresponding numerals in the 400-series. This embodiment features a modular case 402 with a distinctive dome-shaped configuration. The dome-shaped case 402 is specifically designed to optimize the spatial arrangement of the sensor array while ensuring the tibial insert maintains its mechanical integrity under normal loading conditions.

In this embodiment, the radius of the dome, denoted by R2, decreases at the same rate as the radius of the upper portion of the tibial insert, denoted by R1, as shown in FIG. 7. This synchronized reduction in the radii of the dome and tibial insert allows for a smooth and seamless fit between the two components. This design not only ensures that the case 402 fits securely within the tibial insert 400 but also maintains the necessary structural properties of the implant. As with the previously discussed tibial inserts, this embodiment maintains a minimum thickness of 2 mm at the boundary of the articular surface and the tangent point of the domed case 402. This minimum thickness is necessary for ensuring that the tibial insert can both accommodate the sensor array housed within the insert and withstand the mechanical loads imparted during knee joint movement. The 2 mm thickness meets established guidelines for implant durability, ensuring that the tibial insert remains resilient under the compressive, tensile, and shear forces that occur during activities such as walking, running, and climbing stairs.

The dome-shaped case 402 is configured to house a sensor array, which may include various types of sensors such as Hall effect sensors, load sensors, temperature sensors, and more, as described earlier. The case is positioned within a slot in the tibial insert, providing ample space for the sensors while maintaining the necessary protective enclosure. This slot, similar to the slot in tibial insert 200, is strategically placed in an area that is not directly subjected to high loads, ensuring that the functional integrity of the insert is preserved.

The dome shape of case 402 is also significant for its biomechanical benefits. By aligning the decreasing radius of the dome with the natural curvature of the tibial insert, the case is able to distribute the mechanical stresses experienced by the tibial insert more evenly. This design reduces the likelihood of stress concentrations that could lead to material fatigue or failure over time. The smooth, rounded surfaces of the domed case also minimize the risk of friction or wear between the case and the surrounding implant materials, further enhancing the longevity of the implant.

While a knee joint implant is disclosed above, all or any of the aspects of the present disclosure can be used with any implant such as hip implant, shoulder implant, spinal implant, intramedullary nail, bone plate, bone screw, external fixation device, interference screw, etc. Although, the present disclosure generally refers to implants, the systems and method disclosed above can be used with trials to provide real time information related to trial performance. While sensors disclosed above are generally located in the tibial implant (tibial insert) of the knee joint implant, the sensors can be located within the femoral implant in other embodiments. Sensor shape, size and configuration can be customized based on the type of implant and patient-specific needs.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. A knee implant comprising:
a femoral implant configured to be coupled to a femur, and
a tibial implant configured to be coupled to a tibia, the tibial implant includes a tibial insert disposed between the femoral implant and a tibial baseplate, the tibial insert comprising:
an articular surface configured to contact a corresponding femoral articular surface;
at least one sensor and a battery disposed within a void of the tibial insert, and
a detachable case configured to seal an opening of the void,
wherein a thickness of the tibial insert between the detachable case and the articular surface of the tibial insert is 2 mm or more.

2. The knee implant of claim 1, wherein a thickness of the tibial insert between the void and the articular surface of the tibial insert is 2 mm or more.

3. The knee implant of claim 1, wherein a proximal portion of the case defines a first radius and the articular surface of the tibial insert defines a second radius, the first radius being proportional to the second radius.

4. The knee implant of claim 1, wherein the at least one sensor and the battery are disposed within the void in a central region of the tibial insert between a medial central region and a lateral central region.

5. The knee implant of claim 1, wherein the at least one sensor includes a Hall sensor and the femoral implant includes a magnet, the Hall sensor configured to track a location of the magnet.

6. The knee implant of claim 5, wherein the at least one sensor includes a plurality of sensors, the plurality of sensors including at least one load sensor.

7. The knee implant of claim 6, wherein the plurality of sensors include a temperature sensor, a pressure sensor, and a pH sensor.

8. The knee implant of claim 7, wherein the at least one battery includes a plurality of batteries.

9. The knee implant of claim 8, wherein the tibial insert further includes a printed circuit board assembly, a processor, a charging coil, and an antenna located away from a medial central region and a lateral central region.

10. The knee implant of claim 1, wherein the detachable case is configured to hermetically seal the opening.

11. A tibial implant comprising:
a tibial baseplate configured to contact a tibia, and a a tibial insert configured to contact a femoral implant, the tibial insert comprising:
an articular surface configured to contact a corresponding femoral articular surface of the femoral implant;
at least one sensor and a battery disposed within a void of the tibial insert, and
a detachable case configured to seal an opening of the void,
wherein a thickness of the tibial insert between the detachable case and the articular surface of the tibial insert is 2 mm or more.

12. The tibial implant of claim 11, wherein a thickness of the tibial insert between the void and the articular surface of the tibial insert is 2 mm or more.

13. The tibial implant of claim 11, wherein a proximal portion of the case defines a first radius and the articular surface of the tibial insert defines a second radius, the first radius being proportional to the second radius.

14. The tibial implant of claim 11, wherein the at least one sensor and the battery are disposed within the void in a central region of the tibial insert between a medial central region and a lateral central region.

15. The tibial implant of claim 11, wherein the at least one sensor includes a Hall sensor configured to track a location of a magnet of the femoral implant.

16. The tibial implant of claim 15, wherein the at least one sensor includes a plurality of sensors, the plurality of sensors including at least one load sensor.

17. The tibial implant of claim 16, wherein the plurality of sensors include a temperature sensor, a pressure sensor, and a pH sensor.

18. The tibial implant of claim 17, wherein the at least one battery includes a plurality of batteries.

19. The tibial implant of claim 18, wherein the tibial insert further includes a printed circuit board assembly, a processor, a charging coil, and an antenna located away from a medial central region and a lateral central region.

20. The tibial implant of claim 11, wherein the detachable case is configured to hermetically seal the opening.
